(19) United States
(12) Patent Application Publication
(10) Pub. No.: US 2010/0286520 A1
(43) Pub. Date: Nov. 11, 2010

(54) ULTRASOUND SYSTEM AND METHOD TO DETERMINE MECHANICAL PROPERTIES OF A TARGET REGION

(75) Inventors: CHRISTOPHER ROBERT HAZARD, NISKAYUNA, NY (US); YING FAN, NISKAYUNA, NY (US); KENNETH WAYNE RIGBY, CLIFTON PARK, NY (US)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY, SCHENECTADY, NY (US)

(21) Appl. No.: 12/463,854

(22) Filed: May 11, 2009

(51) Int. Cl. A61B 8/00; A61N 7/00

(52) U.S. Cl. 600/439; 601/2

(57) ABSTRACT

An ultrasound system that is configured to determine whether adipose tissue of a patient received therapy at a treatment location. The system includes an ultrasound probe and a shear-wave-generating module to control the probe to provide a shear-wave beam that is configured to generate a shear wave at a first site within the patient. The shear wave is configured to propagate through the treatment location toward a second site within the patient. The system also includes a tracking module to control the probe to track the shear wave at the second site within the patient to determine if the treatment location received the therapy.

### CROSS-REFERENCE TO RELATED APPLICATIONS

This application includes subject matter that is similar to the subject matter described in U.S. Patent Application having Attorney Docket No. 235594 (555-0003US), entitled “ULTRASOUND SYSTEM AND METHOD TO AUTOMATICALLY DELIVER THERAPY BASED ON USER DEFINED TREATMENT SPACES,” and Attorney Docket No. 235615 (555-0004US), entitled “ULTRASOUND SYSTEM AND METHOD TO AUTOMATICALLY IDENTIFY AND TREAT ADIPOSE TISSUE,” both of which are filed contemporaneously herewith and are incorporated by reference in their entirety.

### BACKGROUND OF THE INVENTION

The subject matter herein relates generally to ultrasound therapy systems that provide treatment of a region of interest in a patient, and more particularly, to ultrasound systems that treat adipose tissue.

Various body contouring systems exist today that attempt to remove or destroy fatty tissue (or adipose tissue) from a person's body. Some systems may be invasive, such as liposuction, where a device is inserted into the body and physically removes adipose tissue through suction. Other systems may be non-invasive. For example, in one non-invasive system, high-intensity focused ultrasound (HIFU) signals are directed toward a target region within the adipose tissue. The HIFU signals may at least partially liquefy the adipose tissue at the target region through lysis, cavitation, and/or thermal damage of the cells.

In some known HIFU systems, an operator typically directs several applications of therapy to separate locations within a region of the patient's body. When an operator moves the target from one location in the adipose tissue to a new location in the adipose tissue, the mechanical properties of the adipose tissue in the new location may be different from previous locations. As such, the therapy parameters for delivering the HIFU signals may need to be changed in light of the different mechanical properties. However, known HIFU systems have limited capabilities in identifying the mechanical properties of the target region. Furthermore, known HIFU systems may not be capable of determining whether the therapy delivered to one location was effective in at least partially liquefying the adipose tissue.

Accordingly, there is a need for an ultrasound therapy system that determines mechanical properties of a proposed target region. There is also a need for an ultrasound system that confirms that an effective amount of treatment was delivered to a location within a patient.

### BRIEF DESCRIPTION OF THE INVENTION

In one embodiment, an ultrasound system is provided that is configured to determine whether adipose tissue of a patient received therapy at a treatment location. The system includes an ultrasound probe and a shear-wave-generating module to control the probe to provide a shear-wave beam that is configured to generate a shear wave at a first site within the patient. The shear wave is configured to propagate through the treatment location toward a second site within the patient. The system also includes a tracking module to control the probe to track the shear wave at the second site within the patient to determine if the treatment location received the therapy.

In another embodiment, a method to determine whether adipose tissue of a patient received therapy at a treatment location is provided. The method includes providing a shear-wave beam to generate a shear wave at a first site within the patient. The shear wave is configured to propagate through the treatment location toward a second site within the patient. The method also includes tracking the shear wave at the second site within the patient to determine if the treatment location received the therapy.

In yet another embodiment, a system configured to determine whether adipose tissue of a patient received therapy at a treatment location is provided. The system includes a shear-wave-generator that is configured to generate a shear wave at a first site within the patient. The shear wave is configured to propagate through the treatment location toward a second site within the patient. The system also includes a tracker that is configured to track the shear wave at the second site within the patient to determine if the treatment location received the therapy.

### BRIEF DESCRIPTION OF THE DRAWINGS

FIG. 1 is a schematic diagram of an ultrasound therapy system formed in accordance with an embodiment of the invention.

FIG. 2 is a block diagram of an ultrasound system formed in accordance with an embodiment of the invention.

FIG. 3 is a block diagram of a diagnostic module in the ultrasound system of FIG. 2 formed in accordance with an embodiment of the invention.

FIG. 4 is a block diagram of a therapy module in the ultrasound system of FIG. 2 formed in accordance with an embodiment of the invention.

FIG. 5 illustrates transducer arrays that may be used with a probe in accordance with various embodiments.

FIG. 6 is a schematic diagram of a probe in accordance with an embodiment of the invention and a region-of-interest (ROI) within a patient.

FIG. 7 is a schematic diagram of the ROI in FIG. 6 after therapy has been applied by the probe.

FIG. 8 illustrates another method in accordance with an embodiment of the invention.

FIG. 9 illustrates another method in accordance with an embodiment of the invention.

FIG. 10 illustrates a window presented on a display that displays a treatment space of an ROI.

FIG. 11 illustrates another window presented on the display of FIG. 10 that displays a treatment space of an ROI.

FIG. 12 shows the window in FIG. 10 as the ultrasound system delivers therapy to the treatment space.

FIG. 13 illustrates an ultrasound system in accordance with one embodiment that includes a tracking system and a registering system.

FIG. 14A is a flowchart illustrating a method in accordance with one embodiment.

FIG. 14B is a flowchart illustrating another method in accordance with one embodiment.

FIG. 15 illustrates a hand carried or pocket-sized ultrasound imaging system that may be configured to display a region of interest during a therapy session in accordance with various embodiments.

FIG. 16 illustrates a console-based ultrasound imaging system provided on a movable base that may be configured to display a region of interest during a therapy session in accordance with various embodiments.

FIG. 17 is a block diagram of exemplary manners in which embodiments of the invention may be stored, distributed, and installed on computer readable medium.

### DETAILED DESCRIPTION OF THE INVENTION

Exemplary embodiments that are described in detail below include ultrasound systems and methods for treating a region of interest (ROI). The ROI may include at least one of adipose tissue and non-adipose tissue, such as a dermis layer, muscle tissue, bone, tissue of organs, and blood vessels. The system may also include a display or imaging device that displays the ROI so that an operator or user of the system can distinguish the adipose tissue and the non-adipose tissue and/or the system may automatically differentiate the adipose tissue and the non-adipose tissue prior to treating. Furthermore, the display may also indicate those locations within the ROI that have been treated. Treatment of the ROI may include providing high-intensity focused ultrasound (HIFU) signals to treatment locations within the ROI. When therapy is delivered to a treatment location within adipose tissue, mechanical properties or characteristics of the adipose tissue may be affected. For example, HIFU signals may be directed to treatment locations within the adipose tissue to at least partially liquefy the adipose tissue.

Exemplary embodiments may also include methods and systems for determining whether an effective amount of therapy has been received at a treatment location. As an example, a treatment location within adipose tissue of a patient has received an “effective amount” of therapy if the adipose tissue at the treatment location has been at least partially liquefied for the purpose of body contouring and/or weight loss.

Also, methods and systems described herein may be configured to track a shear wave that propagates through a tissue in order to determine the mechanical properties of the tissue. In particular embodiments, the shear wave is configured to propagate through a treatment location that may or may not have already received therapy. As used herein, a shear wave or waves “propagating through” a site or location may include the shear wave being generated at that site or location or being generated at another adjacent site. More specifically, in some embodiments, therapy may be applied to a treatment location and shear waves may be generated at the treatment location (whether subsequent to or during the application of therapy). The shear waves may be tracked at another site to determine the mechanical properties of the adipose tissue at the treatment location. If the treatment location received an effective amount of therapy, the shear wave may not be detected at the other site or the shear wave may be substantially affected (i.e., if the waveform characteristics of the shear wave that are detected at the other site after therapy are significantly different from the waveform characteristics of the shear wave before therapy).

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It should be noted that although the various embodiments may be described in connection with an ultrasound system, the methods and systems described herein are not limited to imaging and therapy that are exclusively performed through ultrasound. In particular, the various embodiments may be implemented in connection with different types of medical imaging, including, for example, magnetic resonance imaging (MRI) and computed-tomography (CT) imaging. In addition, shear waves may be generated by an ultrasound probe or by another system or device. Further, the various embodiments may be implemented in other non-medical imaging systems, for example, non-destructive testing systems, such as airport screening systems.

A technical effect of the various embodiments of the systems and methods described herein include confirming or determining if a treatment location within adipose tissue has been at least partially liquefied. Another technical effect may include determining mechanical properties of adipose tissue at a target region or a treatment location. Another technical effect may include generating an image of an ROI and indicating to a user that a treatment location within the ROI has been at least partially liquefied. Another technical effect may include providing therapy to treatment locations and automatically moving the treatment location between multiple points (or treatment sites) within the treatment. Other technical effects may be provided by the embodiments described herein.

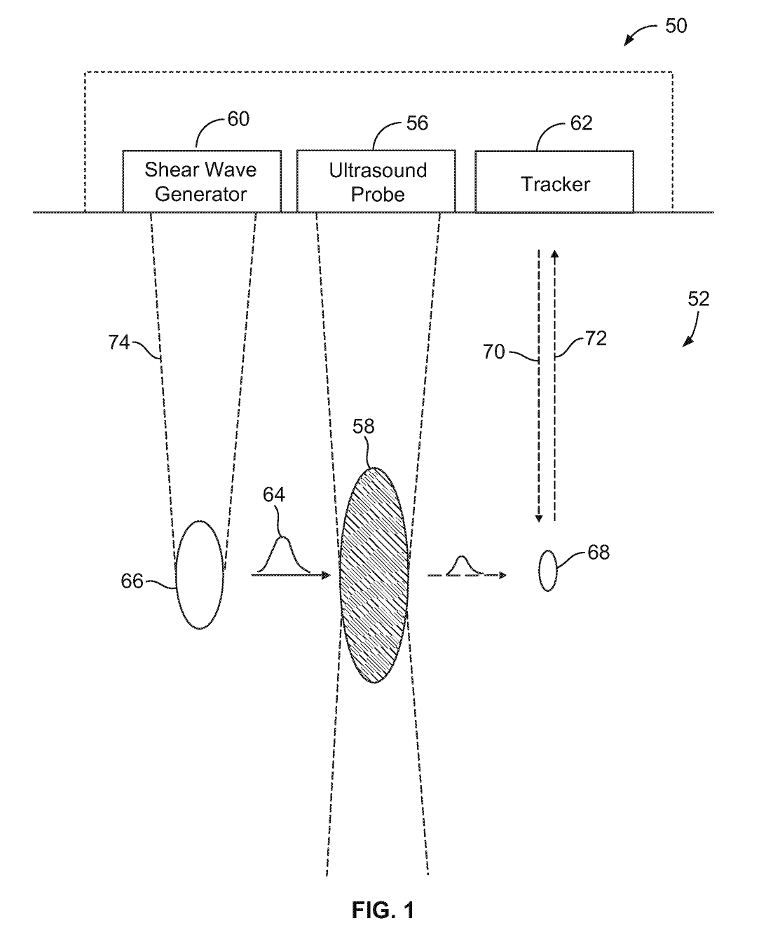

FIG. 1 is a diagram of an exemplary ultrasound system 50 formed in accordance with an embodiment. The system 50 is configured to treat adipose tissue 52 (or another tissue) within a region of interest (ROI) of a patient and/or determine mechanical properties of the adipose tissue 52 (or the other tissue) within the ROI. The system 50 may include an ultrasound probe 56, a shear-wave generator 60, and a tracker 62. The probe 56 is configured to deliver, during a therapy session, a therapy to a treatment location 58 within the adipose tissue 52 of the ROI. For example, the probe 56 may be configured to deliver HIFU signals to the treatment location 58. The therapy delivered to the treatment location 58 may affect mechanical properties of the adipose tissue 52 at the treatment location 58. For instance, the adipose tissue 52 at the treatment location 58 may be lysed through cavitation or thermal treatment resulting in the adipose tissue 52 being at least partially liquefied. The liquefied adipose tissue 52 exhibits different mechanical properties than adipose tissue 52 that has not received the therapy. The resulting change, if any, in the mechanical properties at the treatment location 58 may facilitate determining (a) whether the therapy was received by the adipose tissue at the treatment location 58; (b) whether the therapy was effective for treating the adipose tissue 52; and (c) information regarding the mechanical properties of the adipose tissue 52 at the treatment location 58.

The system 50 may determine the mechanical properties of the treatment location 58 before and after treatment using elastographic methods and, particularly, using shear waves. Mechanical properties may include at least one of a shear elasticity modulus, Young's modulus, dynamic shear viscosity, and mechanical impedance of the target tissue. The mechanical properties may be calculated or determined using propagation parameters, such as a shear wave velocity, a shear wave attenuation coefficient, an amplitude and velocity of shear displacement of tissue particles in a propagating shear wave, and spatial and temporal dependencies of the amplitude and velocity of shear displacement of tissue particles. Another parameter that may be used is shear viscosity, which may be derived from changes in shear velocity as a function of frequency.

As shown, the treatment location 58 is located substantially between a generation site 66 (also called a first site or a shear-wave source) and a tracking site 68 (also called a second site). Prior to generating a shear wave, the tracker 62 may deliver one or more tracking pulses 70 toward the tracking site 68. A reflection signal 72 from the adipose tissue 52 of the tracking site 68 may be indicative of the adipose tissue 52 at the second site 68 in a baseline condition (i.e., before any displacement caused by the shear wave).

After obtaining the reflection signal(s) 72 of the second site 68 before any displacement, the shear-wave generator 60 is configured to deliver a pushing or shear-wave-generating pulse 74 toward the first site 66 that is proximate to the treatment location 58. The pushing pulse 74 generates a shear wave 64 at the first site 66 that propagates toward the treatment location 58 (also called target region) and toward the second site 68. The shear-wave generator 60 may generate the shear wave 64 before therapy has been applied to the treatment location 58. The tracker 62 is configured to track the shear wave 64 at the second site 68 by delivering one or more tracking pulses 70 toward the second site 68. When the shear wave 64 propagates to the second site 68, another reflection signal 72 of the tracking pulse 70 is provided. More specifically, the tracking pulses 70 are modified by the adipose tissue 52 at the second site 68 while experiencing the shear wave 64. The reflection signal 72 from the second site 68 indicates a reference displaced condition of the adipose tissue 52 at the second site 68. Reflection signals 72 of the reference displaced condition and the baseline condition of the second site 68 provide references that may be compared to by other conditions of the second site 68.

After receiving the reflected signal 72 of the reference displaced condition, the probe 56 may deliver therapy (e.g., HIFU signals) to the treatment location 58. During or subsequent to treatment, the shear-wave generator 60 may deliver one or more pushing or shear-wave-generating pulses 74 to the first site 66 to generate another shear wave 64. If an effective amount of therapy was received by the adipose tissue 52 at the treatment location 58, the mechanical properties of the adipose tissue 52 at the treatment location 58 should substantially affect propagation of the shear wave 64. For example, if the treatment location 58 is substantially liquefied, the shear wave 64 may not propagate through the treatment location 58 or reach the second site 68. If the shear wave 64 reaches the second site 68, characteristics of the shear wave 64 (e.g., velocity and amplitude) may be significantly affected. More specifically, the velocity may be slower and the amplitude may be smaller as compared to a velocity and amplitude of the shear wave 64 when the treatment location 58 has not received therapy.

The pushing pulse 74 and the tracking pulse 70 may be delivered at different intensity levels. The pushing pulse 74 may be an intense pulse that generates an appreciable radiation force at the first site 66. For example, the pushing pulse 74 may be an amplitude-modulated beam of focused ultrasound that provides a radiation force to generate the shear wave 64 at the first site 66. A significant amount of acoustic energy may be delivered to the first site 66 to achieve a desired initial displacement at the first site 66. The localized force generating the shear wave 64 causes tissue displacement along the wave path. In some embodiments, the shear waves may propagate from a point source. In other embodiments, the shear waves may be virtually extended shear waves extending along a plane.

The tracker 62 may use any imaging technique capable of detecting internal motion of tissue within the ROI. For example, tissue displacements at the first, second, or any other site may be determined using ultrasonic correlation based methods or other pattern matching methods. The tracker 62 may use any pulsed-echo imaging methods, B-mode imaging methods, and pulsed or continuous Doppler imaging methods. Furthermore, the tracker 62 may be a magnetic resonance imaging (MRI) system. Optical tracking may also be used to detect internal motion of the ROI.

Separate components or devices may be used for therapy, imaging, shear wave generation, and shear wave detection. Alternatively, one component or device (e.g., a transducer) may be used for any combination (or all) of therapy, imaging, shear-wave-generating, and detecting. Accordingly, as shown in FIG. 1, the shear-wave generator 60, the probe 56, and the tracker 62 may all be incorporated into a single device or system, such as the system 120 described in detail below. More specifically, the functions of the shear-wave generator 60 and the tracker 62 may be performed by the probe 56. However, in alternative embodiments, one or more of the shear-wave generator 60, the probe 56, and the tracker 62 operates separately from the other components. For example the shear-wave generator 60 and the tracker 62 may be separate ultrasound probes. Also, in alternative embodiments, the shear-wave generator may be made by a mechanical actuator or an audio speaker.

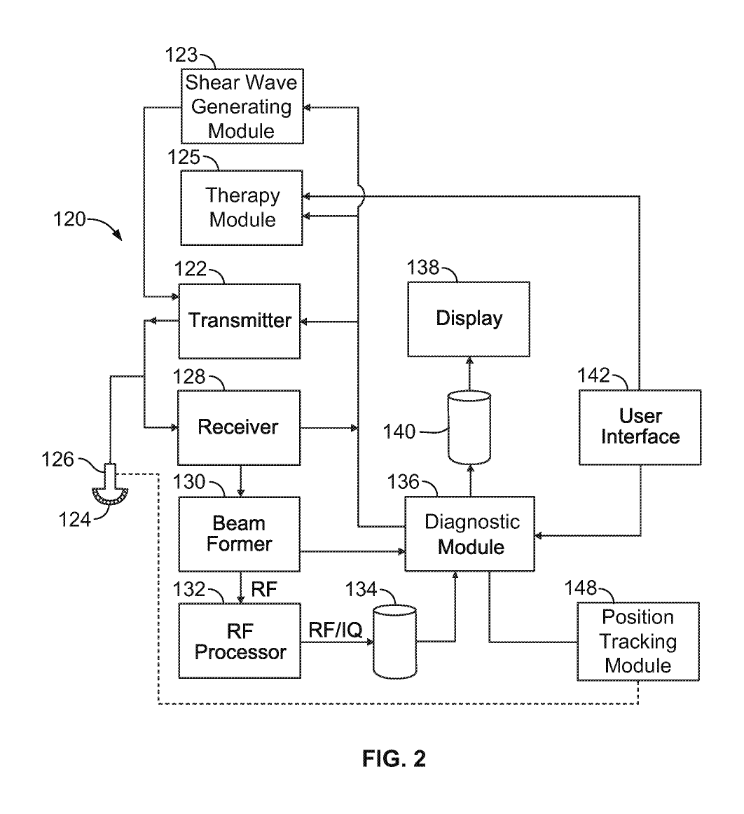

FIG. 2 is a block diagram of an exemplary ultrasound therapy system 120 in which various embodiments can provide therapy to and/or display a ROI during a therapy session for treating adipose tissue. The ultrasound system 120 includes a transmitter 122 that drives an array of transducer elements 124 (e.g., piezoelectric crystals) within a probe 126 to emit pulsed ultrasonic signals into a body or volume. The pulsed ultrasonic signals may be for imaging and/or for therapy of tile ROI. The pulsed ultrasonic signals may also be for generating and/or tracking shear waves. For example, the probe 126 may deliver low energy pulses during imaging and tracking, medium to high energy pulses to generate shear waves, and high energy pulses during therapy. A variety of array geometries and configurations may be used and the array of transducer elements 124 may be provided as part of, for example, different types of ultrasound probes.

The imaging and tracking signals may be back-scattered from structures in the body, for example, adipose tissue, muscular tissue, connective tissue, blood cells, veins or objects within the body (e.g., a catheter or needle) to produce echoes that return to the elements 124. The echoes are received by a receiver 128. The received echoes are provided to a beamformer 130 that performs beamforming and outputs an RF signal. The RF signal is then provided to an RF processor 132 that processes the RF signal. Alternatively, the RF processor 132 may include a complex demodulator (not shown) that demodulates the RF signal to form IQ data pairs representative of the echo signals. The RF or IQ signal data may then be provided directly to a memory 134 for storage (e.g., temporary storage). Optionally, the output of the beamformer 130 may be passed directly to the diagnostic module 136.

The ultrasound system 120 also includes a processor or diagnostic module 136 to process the acquired ultrasound information (e.g., RF signal data or IQ data pairs) and prepare frames of ultrasound information for display on a display 138. The diagnostic module 136 is adapted to perform one or more processing operations according to a plurality of selectable ultrasound modalities on the acquired ultrasound information. Acquired ultrasound information may be processed in real-time during a scanning or therapy session as the echo signals are received. Additionally or alternatively, the ultrasound information may be stored temporarily in the memory 134 during a scanning session and processed in less than real-time in a live or off-line operation. An image memory 140 is included for storing processed frames of acquired ultrasound information that are not scheduled to be displayed immediately. The image memory 140 may comprise any known data storage medium, for example, a permanent storage medium, removable storage medium, etc.

The diagnostic module 136 is connected to a user interface 142 that controls operation of the diagnostic module 136 as explained below in more detail and is configured to receive inputs from a user. The display 138 includes one or more monitors that present patient information, including diagnostic and therapeutic ultrasound images to the user for review, diagnosis, analysis, and treatment. The display 138 may automatically display, for example, a 2D, 3D, or 4D ultrasound data set stored in the memory 134 or 140 or currently being acquired, which data set is also displayed with a graphical representation (e.g., an outline of a treatment space or a marker within the treatment space). One or both of the memory 134 and the memory 140 may store 3D data sets of the ultrasound data, where such 3D data sets are accessed to present 2D and 3D images. For example, a 3D ultrasound data set may be mapped into the corresponding memory 134 or 140, as well as one or more reference planes. The processing of the data, including the data sets, may be based in part on user inputs, for example, user selections received at the user interface 142.

The diagnostic module 136 is configured to analyze ultrasound signals for imaging the ROI and/or tracking shear waves propagating through the ROI. Furthermore, the diagnostic module 136 may also automatically differentiate adipose tissue from non-adipose tissue (e.g., muscle tissue, bone, connective tissue, organs). The diagnostic module 136 may also be configured to receive user imaging commands for outlining or otherwise providing an overlay that indicates a treatment space within the ROI. The diagnostic module 136 may also receive user therapy commands (e.g., through the user interface 142) regarding how to apply therapy to treatment locations within the ROI. The therapy commands may relate to therapy parameters and the like.

The diagnostic module 136 communicates with a therapy module 125 that is configured to control the probe 126 during a therapy session. A “therapy session,” as used herein, is a period of time in which a patient receives therapy. For example, a therapy session may include a single application of ultrasounds signals to liquefy adipose tissue at a single treatment location or within a single treatment space within the body. A therapy session may also include an extended period of time in which a patient receives multiple applications of ultrasound signals within a treatment space of one region of the body or within multiple regions of the body. A therapy session may also include one visit by a patient to an operator of the system 120.

The diagnostic module 136 may be configured to control the probe 126 to deliver and obtain diagnostic ultrasound signals from the ROI and to control the probe 126 to deliver a pushing pulse to generate a shear wave within the ROI. The therapy module 125 is configured to deliver a therapy to the treatment locations based within the ROI. The therapy module 125 may automatically move the treatment location between multiple points based on user inputs.

In operation, the system 120 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, etc.). The data may be acquired by moving the probe 126, such as along a linear or curvilinear path, while scanning the ROI. At each linear or arcuate position, the probe 126 obtains scan planes that are stored in the memory 134. The probe 126 also may be mechanically moveable within the ultrasound probe.

The system 120 also includes a shear-wave-generating module 123 that is operatively coupled to the diagnostic module 136 or a sub-module of the diagnostic module 136. The shear-wave-generating module 123 is configured to control the probe 126 to generate a shear wave at a site within the ROI of the patient. The shear-wave-generating module 123 may control the probe 126 or, more particularly, the transducer elements 124 to direct a shear-wave-generating or pushing pulse(s) toward the predetermined site to generate the shear wave. Alternatively, the shear-wave-generating module 123 may control another device capable of generating shear waves. For example, the shear-wave-generating module 123 may control a therapy transducer, a mechanical actuator, or an audio device to generate the shear waves. The pushing pulse(s) may be configured to generate a desired shear wave and to direct the shear wave along a desired path or direction.

Optionally, the system 120 may include a position tracking module 148 that tracks a position of the probe 126 and communicates the position to the diagnostic module 136. A position of the probe 126 may be tracked relative to a reference point on or near the patient, a marker, and the like. As will be described in greater detail below, the position of the probe 126 may be used to indicate, to the user, regions of the patient that have already been treated, are being treated, or have yet to be treated.

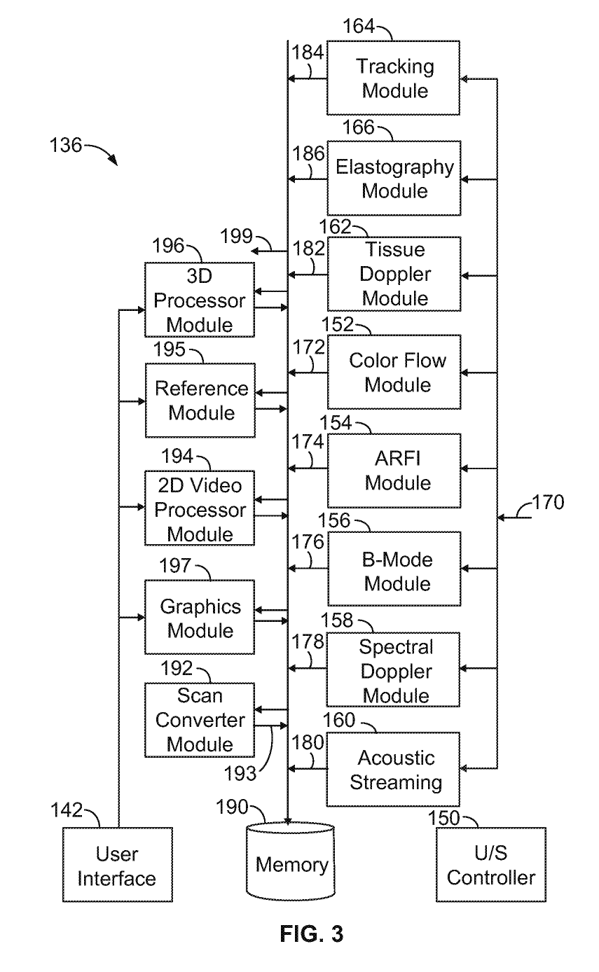

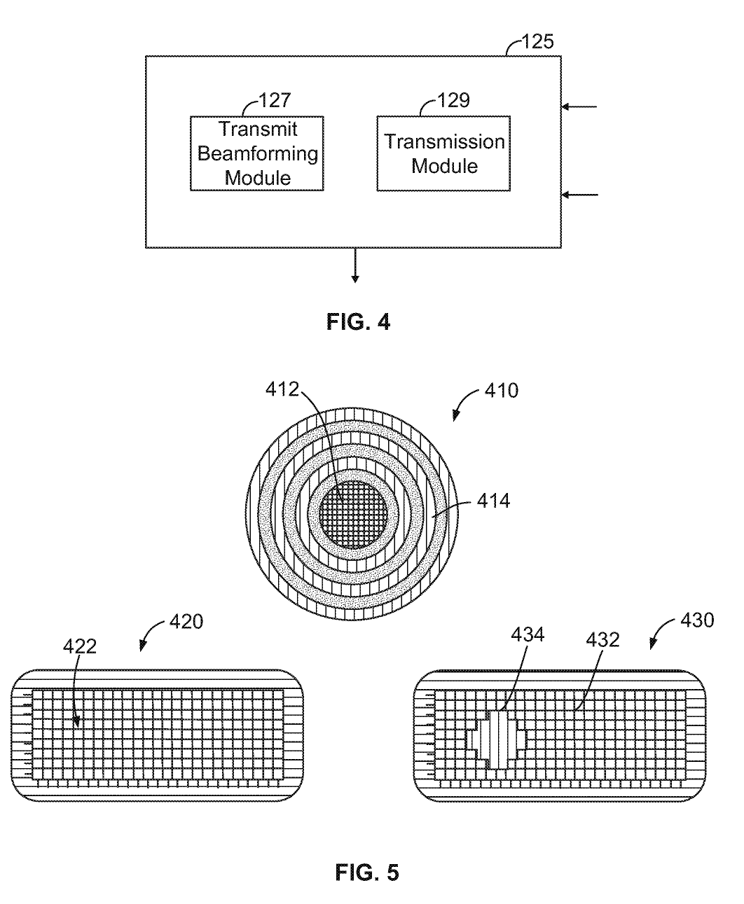

FIG. 3 is an exemplary block diagram of the diagnostic module 136, and FIG. 4 is an exemplary block diagram of the therapy module 125. The therapy and diagnostic modules 125 (FIG. 4) and 136 (FIG. 3) are illustrated conceptually as a collection of modules, but may be implemented utilizing any combination of dedicated hardware boards, DSPs, processors, etc. Alternatively, the modules of FIGS. 3 and 4 may be implemented utilizing an off-the-shelf PC with a single processor or multiple processors, with the functional operations distributed between the processors. As a further option, the modules of FIGS. 3 and 4 may be implemented utilizing a hybrid configuration in which certain modular functions are performed utilizing dedicated hardware, while the remaining modular functions are performed utilizing an off-the-shelf PC and the like. The modules also may be implemented as software modules within a processing unit. Furthermore, the diagnostic module 136 may include the therapy module 125, or the therapy module 125 may include the diagnostic module 136.

The operations of the modules illustrated in FIGS. 3 and 4 may be controlled by a local ultrasound controller 150 or by the diagnostic module 136. The modules 152-166 perform mid-processor operations. The diagnostic module 136 may receive ultrasound data 170 in one of several forms. In the embodiment of FIG. 3, the received ultrasound data 170 constitutes IQ data pairs representing the real and imaginary components associated with each data sample. The IQ data pairs are provided to one or more modules, for example, a color-flow module 152, an acoustic radiation force imaging (ARFI) module 154, a B-mode module 156, a spectral Doppler module 158, an acoustic streaming module 160, a tissue Doppler module 162, a tracking module 164, and an elastography module 166. Other modules may be included, such as an M-mode module, power Doppler module, among others. However, embodiments described herein are not limited to processing IQ data pairs. For example, processing may be done with RF data and/or using other methods. Furthermore, data may be processed through multiple modules.

Each of modules 152-166 is configured to process the IQ data pairs in a corresponding manner to generate, respectively, color-flow data 172, ARFI data 174, B-mode data 176, spectral Doppler data 178, acoustic streaming data 180, tissue Doppler data 182, tracking data 184, elastography data 186, among others, all of which may be stored in a memory 190 (or memory 134 or image memory 140 shown in FIG. 2) temporarily before subsequent processing. The data 172-186 may be stored, for example, as sets of vector data values, where each set defines an individual ultrasound image frame. The vector data values are generally organized based on the polar coordinate system.

A scan converter module 192 accesses and obtains from the memory 190 the vector data values associated with an image frame and converts the set of vector data values to Cartesian coordinates to generate an ultrasound image frame 193 formatted for display. The ultrasound image frames 193 generated by the scan converter module 192 may be provided back to the memory 190 for subsequent processing or may be provided to the memory 134 (FIG. 2) or the image memory 140 (FIG. 2). Once the scan converter module 192 generates the ultrasound image frames 193 associated with the data, the image frames may be restored in the memory 190 or communicated over a bus 199 to a database (not shown), the memory 134, the image memory 140 and/or to other processors (not shown).

As an example, it may be desired to view different ultrasound images relating to a therapy session in real-time on the display 138 (FIG. 2). To do so, the scan converter module 192 obtains data sets for images stored in the memory 190 of that are currently being acquired. The vector data is interpolated where necessary and converted into an X, Y format for video display to produce ultrasound image frames. The scan converted ultrasound image frames are provided to a display controller (not shown) that may include a video processor that maps the video to a gray-scale mapping for video display. The gray-scale map may represent a transfer function of the raw image data to displayed gray levels. Once the video data is mapped to the gray-scale values, the display controller controls the display 138, which may include one or more monitors or windows of the display, to display the image frame. The image displayed in the display 138 is produced from an image frame of data in which each datum indicates the intensity or brightness of a respective pixel in the display.

Referring again to FIG. 3, a 2D video processor module 194 may be used to combine one or more of the frames generated from the different types of ultrasound information. For example, the 2D video processor module 194 may combine different image frames by mapping one type of data to a gray map and mapping the other type of data to a color map for video display. In the final displayed image, the color pixel data is superimposed on the gray scale pixel data to form a single multi-mode image frame that is again re-stored in the memory 190 or communicated over the bus 199. Successive frames of images may be stored as a cine loop (4D images) in the memory 190 or memory 140 (FIG. 2). The cine loop represents a first in, first out circular image buffer to capture image data that is displayed in real-time to the user. The user may freeze the cine loop by entering a freeze command at the user interface 142. The user interface 142 may include, for example, a keyboard and mouse and all other input controls associated with inputting information into the ultrasound system 120 (FIG. 2). In one embodiment, the user interface 142 includes the display 138 that may be touch-sensitive or configured to interact with a stylus. The user interface 142 may also receive user inputs through voice-recognition or activation.

A 3D processor module 196 is also controlled by the user interface 142 and accesses the memory 190 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel projection and the like.

A graphic module 197 may also be controlled by the system 120 and may access the memory 190 to obtain groups of ultrasound image frames that have been stored or that are currently being acquired. The graphic module 197 may generate images that include the images of the ROI and a graphical representation positioned (e.g., overlaid) onto the images of the ROI. The graphical representation may represent an outline of a treatment space, the focal point or region of the therapy beam, a path taken by the focal region within the treatment space, a probe used during the session, and the like. Graphical representations may also be used to indicate the progress of the therapy session. The graphical representations may be generated using a saved graphical image or drawing (e.g., computer graphic generated drawing), or the graphical representation may be directly drawn by the user onto the image using a pointing device, e.g., an electronic stylus or mouse, or another interface device.

Also shown, a reference module 195 may be used to identify a reference point on or near the patient during the therapy session. For example, a reference point may be an anatomical element or structure of the body that is determined by the system 120 or by the user. The reference point may also be an element or marker positioned on the surface of the body of the patient. As will be described in greater detail below, the reference module 195 may use the imaging data to determine a relation of the treatment space with respect to a reference point.

Referring to FIG. 4, the therapy module 125 may be coupled to the diagnostic module 136 (FIG. 2) and the user interface 142 (FIG. 2) and include a transmit beamforming module 127 and a transmission module 129. The transmit beamforming module 127 is configured to control the location and movement of a focal point or region generated by the transducer elements 124. For example, the transmit beamforming module 127 may control electronic or mechanical steering of the probe to move the focal region of a therapy beam within the treatment spaces or between different treatment spaces. The transmission module 129 is configured to drive the transducer elements 124 (or only a portion or subset of the transducer elements 124) in delivering energy pulses to the ROI for imaging and therapy.

FIG. 5 illustrates transducers 410, 420, and 430 that may be used with a probe (not shown) in accordance with various embodiments. The transducers 410, 420, and 430 may include reconfigurable arrays. In some embodiments, the diagnostic module 136 (FIG. 2) controls the probe 126 (FIG. 2) to deliver imaging and/or tracking pulses, the shear-wave-generating module 123 (FIG. 2) controls the probe to generate a shear wave, and the therapy module 125 (FIG. 2) controls the probe to generate therapy pulses. Relative to one another, the imaging and tracking pulses may be low energy, the shear-wave-generating pulses may be medium to high energy, and the therapy pulses may be high energy.

The transducer 410 has an imaging array 412 and a separate therapy array 414 that surrounds the imaging array 412. The imaging array 412 and the therapy array 414 may be in a fixed relationship with respect to each other. The imaging pulses and the therapy pulses may be delivered separately or in an overlapping or interleaved manner. In some embodiments, low energy imaging pulses and shear-wave-generating pulses may also be transmitted by the therapy array 414. The reflections of these low energy imaging pulses may be received by the imaging array 412 to form an image of the focal region of the therapy array 414 or to track a shear wave. Embodiments of a dual therapy and imaging system are described in more detail in U.S. Pat. No. 5,769,790, which is incorporated by reference in the entirety.

The transducer 420 includes an array 422 where the entire array may be used for imaging, tracking, and therapy. However, the transducer 430 has an array 432 of transducer elements where a therapy portion 434 of the array 432 may be activated to provide therapy. As such, the therapy module 125 may drive a subset (e.g., the therapy portion 434) of the transducer elements of the array 432 based on the user inputs designating the treatment space. Although not shown, the transducer 430 may also have tracking portion of the array 432 that includes a subset of transducer elements for tracking shear waves. The therapy portion, tracking portion, and an imaging portion may share transducer elements. Thus, the diagnostic module 136 and the therapy module 125 may deliver low energy imaging and/or tracking pulses and high energy therapy pulses, respectively, in an interspersed manner to an at least partially overlapping array of transducer elements.

When imaging or applying therapy to a patient, the pressure applied by the transducer to the patient's body may alter the thickness or other characteristics of the ROI, such as tissue stiffness. By combining the imaging, therapy, and tracking arrays into one transducer, therapy may be applied immediately after the transducer images the ROI and the shear wave may be immediately generated thereafter and before. As such, an accurate representation or identification of the adipose tissue may be provided immediately before the therapy is applied. However, in alternative embodiments, separate transducers may be used for therapy, imaging, and/or tracking.

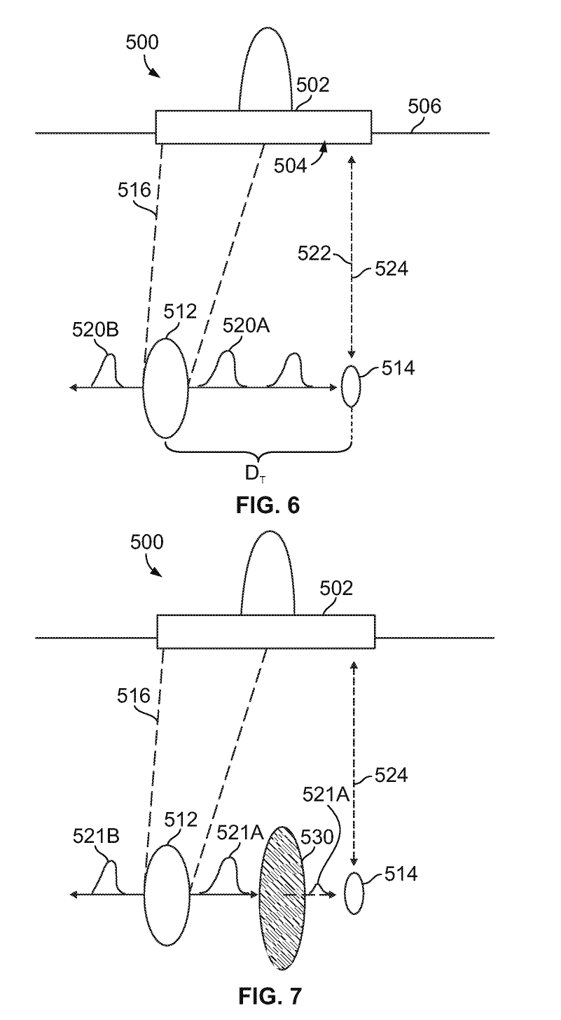

FIGS. 6-9 are schematic diagrams illustrating an ultrasonic probe 500 or methods configured to deliver therapy to a treatment location and to confirm whether an amount of therapy received at the treatment location was sufficient for ablating or liquefying adipose tissue. In addition or alternatively, the ultrasonic probe 500 may be used to determine mechanical properties of the adipose tissue proximate to the treatment location.

FIG. 6 illustrates the probe 500 having a transducer 502 with an array 504 of transducer elements. As shown, the transducer 502 is located on a skin surface 506 on an ROI of a patient. The ROI includes adipose tissue at a depth beneath the surface 506. In some embodiments, the probe 500 may deliver tracking pulses 522 to a tracking or second site 514 to receive reflection signals 524 indicative of a baseline condition of the adipose tissue. Also, before therapy is applied to a treatment location, the probe may generate shear waves 520 at a first or generation site 512 to determine a reference displaced condition of the adipose tissue at the second site 514. The first and second sites 512 and 514 may be separated from each other a distance D_{T}. The transducer 502 may deliver a beam or a pulse 516 of focused ultrasound signals to a predetermined spatial volume at the first site 512 to generate the shear waves 520. The resulting radiation force at the first site 512 generates the shear waves 520A and 520B that propagate away from the first site 512 in opposing directions that are substantially parallel to a surface of the transducer 502. Furthermore, since the propagation of the shear waves 520A and 520B are directly related to mechanical properties of the adipose tissue within the ROI, the distance D_{T} may be configured so that the shear waves 520 are capable of crossing the distance D_{T} without being fully attenuated.

Although FIG. 6 only shows shear waves 520A and 520B traveling perpendicular to a direction of the beam, many shear waves may be generated at the first site 512 that propagate away from the first site in multiple directions. The shear waves may be spherically diverging, diverging from a plane, or diverging in some other configuration. Furthermore, in some embodiments, multiple pushing pulses may be used to create a shear wave directed in a particular direction. Such shear waves may be generated using methods similar to those used in supersonic shear wave imaging.

After delivering the beam 516 for generating the shear waves 520, the transducer 502 may deliver a series of tracking pulses 522 toward the second site 514. When the shear wave 520A propagates through the second site 514, the reflected signals 524 are modified from the baseline condition by the shear wave 520A. The reflected signals 524 from the series of tracking pulses 522 represent various levels of displacement of the adipose tissue as the shear wave 520A or waves 520A pass through the second site 514. A relative motion of the adipose tissue at the second site 514 may be determined using correlations between the reflected signals 524 of the second site 514 in the baseline condition and displaced conditions. For example, a displacement as a function of time for the adipose tissue at the second site 514 may be determined. Accordingly, reference characteristics of the shear wave 520A may be determined. These reference characteristics may later be compared to characteristics of shear waves propagating through treated adipose tissue.

More specifically, the baseline and displaced conditions of the second site 514 may facilitate determining mechanical properties of the adipose tissue at the region between the first and second sites 512 and 514. Furthermore, the baseline and displaced conditions may facilitate determining whether therapy was received by a treatment location located between the first and second sites 512 and 514. Also, a time for propagating the distance D_{T} may be calculated by measuring the time between shear wave generation and shear wave detection.

FIG. 7 illustrates the probe 500 at a time after therapy has been delivered to a treatment location 530. After therapy has been delivered, the transducer 502 may deliver the beam 516 to the first site 512 to generate more shear waves 521. The shear wave 521A may be directed to propagate toward the second site 514 and the shear wave 521B propagates in an opposite direction. When the shear wave 521A propagates through the treatment location 530, the mechanical properties of the adipose tissue at the treatment location 530 may affect the propagation of the shear wave 521A. In particular, if the therapy received at the treatment location 530 has at least partially liquefied the adipose tissue, the propagation of the shear wave 521A may be significantly affected.

The reflected signals 524 may indicate characteristics of the shear wave 521A that are different from the characteristics of the shear wave 520A (FIG. 6) at the second site 514. For example, at least one of the shear wave velocity, the shear wave amplitude, the shear wave viscosity, and the shear wave frequency may be noticeably different at the second site 514 (i.e., values or data of the reflected signals before and after treatment may be significantly different indicating a change in mechanical properties). Furthermore, in some instances, the shear wave 521A may not reach the second site 514 or may not propagate through the treatment location 530 within a predetermined period of time. For example, the shear wave 520 may not propagate through the second site 514 within a predetermined time period that is greater than the time period calculated for a shear wave to propagate the distance D_{T} before treatment.

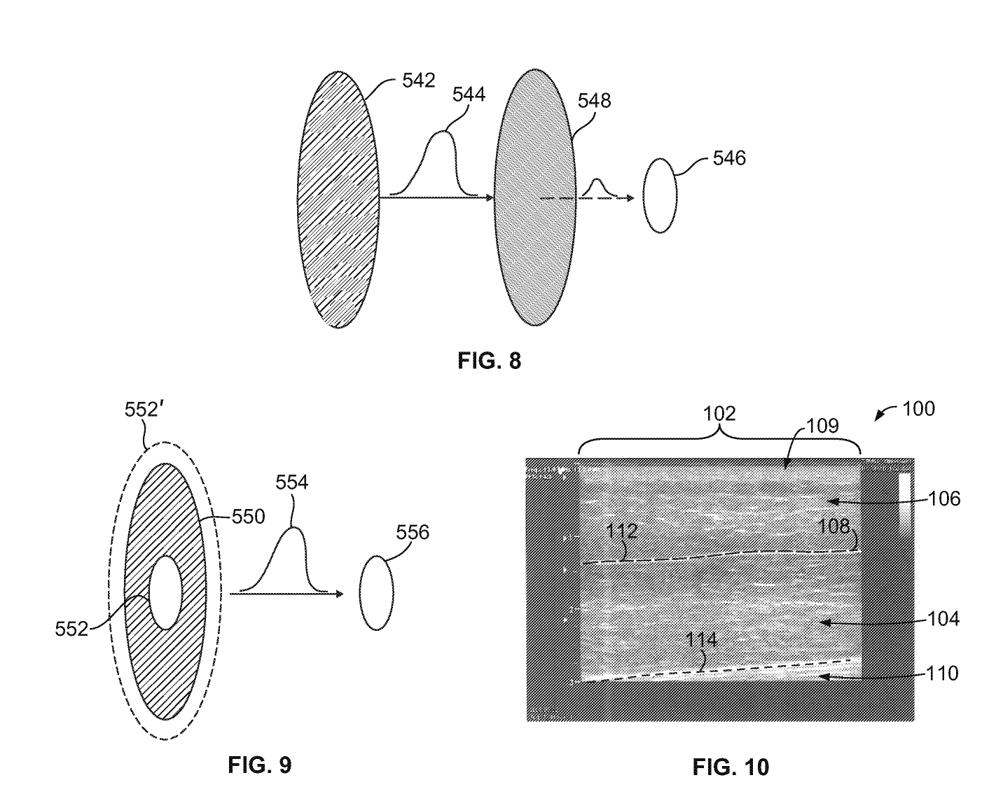

FIG. 8 illustrates another method for confirming that therapy was received at a treatment location. When therapy is applied to a treatment location 542, shear waves 544 may be generated from the treatment location 542 and propagate in a direction toward a tracking site 546. In some embodiments, the therapy system (not shown) may utilize the shear waves 544 generated at the treatment location 542 to determine the effectiveness of therapy applied to other treatment locations. More specifically, as shown, a treatment location 548 may be located substantially between the treatment location 542 and the tracking site 546. The treatment location 548 receives therapy before the treatment location 542. When the therapy is subsequently delivered to the treatment location 542, the shear waves 544 may propagate toward the treatment location 548. Mechanical properties of the adipose tissue at the treatment location 548 may affect the wave propagation of the shear waves 544 as described above. Accordingly, the tracking site 546 may be tracked after therapy is applied to the treatment location 542 to determine if ablation of the adipose tissue at the treatment location 548 was effective.

Alternatively, the shear waves 544 generated at the treatment location 548 may be tracked to determine characteristics or mechanical properties of the adipose tissue proximate to the treatment location 548.

FIG. 9 illustrates another method for confirming that therapy was received at a treatment location. In some embodiments, shear-waves that are configured to be generated at the treatment location may be detected. For example, FIG. 9 shows a treatment location 550 and a first or generation site 552. A first shear-wave generating beam may be directed toward the treatment location 550 before therapy has been applied to the treatment location 550. The first shear-wave generating beam generates shear waves 554 at the first site 552 that at least partially overlaps the intended treatment location 550. The shear waves 554 may be detected at an adjacent tracking site 556. The detected shear waves 554, prior to therapy being applied to the treatment location 550, provide an initial or baseline condition of the tissue at the tracking site 556. Subsequently, a therapy beam may be delivered to the treatment location 550. A second shear-wave generating beam may then be directed toward the treatment location 550. If the treatment location 550 had received an effective amount of therapy, the shear waves 554 will not be detected at the tracking site 556 or will be significantly weaker and/or slower.

As shown in FIG. 9, in some embodiments, dimensions of the treatment location 550 (i.e., an area or volume within the adipose tissue) may be greater than dimensions of the first site 552 (i.e., dimensions of the generation pulse created by the shear-wave generating beam). More specifically, a focal region of the therapy beam may be greater than a focal region of the shear-wave generating beam. As one example, if the shear-wave generating beam has a higher frequency, such as a harmonic, a narrower shear-wave generating beam may be formed such that the generation site 552 is within the treatment location 550 as shown in FIG. 9. In other embodiments, the shear-wave generating beam may have a focal region that is greater than the focal region of the therapy beam such that the first site (indicated by 552′) has larger dimensions than the treatment location 550.

In other embodiments, shear waves may be used to determine a size, shape, or make-up of the treatment location after therapy has been delivered. For example, the shear waves may be separately generated at multiple generation sites on one side of the treatment location along a vertical axis. The shear waves may be detected at respective tracking sites along the vertical axis on the other side of the treatment location. At the center of the treatment location, the adipose tissue may be most affected by the therapy and any shear waves would not be able to propagate therethrough. However, at a periphery of the treatment location, the shear waves may pass through with some additional attenuation. Those shear waves with more attenuation indicate areas of the treatment location that received effectively more therapy. Accordingly, a size, shape, and make-up of the treatment location may be determined. Alternatively, the shear waves may be generated within the treatment location as discussed above, and the shear waves may be detected to determine the mechanical properties of the tissue within the treatment location.

FIG. 10 illustrates a window 100 that may be presented on the display 138 (FIG. 2). The display 138 communicates with the diagnostic module 136 (FIG. 2) to display an image 102 of the ROI of the patient within the window 100. As shown in the image 102, the ROI may include adipose layers or tissues 104 and 106 and non-adipose layers or tissues, such as dermis layer 109 or connective tissue 108, and muscle tissue 110. The image 102 of the ROI may also include other anatomical structures such as bone, organs, cartilage, and others. As will be discussed in greater detail, the system 120 may be able to automatically identify or differentiate between the layers. After differentiating the adipose tissue 104 from other tissues, the system 120 may deliver therapy pulses (e.g., HIFU) to treatment locations within the adipose tissue 104. The system 120 may also confirm that an effective amount of therapy was received at the treatment locations by using, for example, the shear wave methods described above. Alternatively, the user of the system 120 may be able to recognize or identify through the image 102 the different layers of tissue and designate certain locations to receive therapy. The user may also be able to visually identify whether or not the therapy was effective.

In order to automatically differentiate the adipose tissue 104, 106 from the non-adipose tissues and, more specifically, the dermis layer 109, connective tissue 108, and muscle tissue 110, the diagnostic module 136 may analyze ultrasound signals received from the ROI. For instance, the diagnostic module 136 may use data obtained through one or more processing methods (e.g., B-mode, elastography, color-flow). In some embodiments, the diagnostic module 136 automatically differentiates the adipose tissue 104 from the non-adipose tissues by at least identifying barriers or boundaries 112 and 114 (indicated as hashed lines) between the adipose tissues 104 and 106 and/or an adjacent non-adipose tissue, such as the dermis layer 109 and the muscle tissue 110. For example, the diagnostic module 136 may identify the connective tissue 108 that extends between the adipose tissues 104 and 106. The system 120 (FIG. 2) may use stored information regarding general human anatomy at the ROI to identify a location of the adipose tissue 104 in relation to the boundary 112 (i.e., the connective tissue 108 shown in FIG. 10). More specifically, the system 120 may identify the adipose tissue 104 as the tissue or layer extending directly beneath the connective tissue 108. Optionally, the system 120 may indicate to the operator the different layers and tissues within the ROI by illustrating graphical representations of the barriers or boundaries 112 and 114, such as overlaying hashed lines onto the image 102 as shown in FIG. 10.

The system 120 may automatically differentiate tissues within the ROI using other methods separately or in conjunction with identifying barriers or boundaries. As another example, the diagnostic module 136 may automatically differentiate the adipose tissue 104 and 106 from other tissues by at least directly measuring a plurality of points within the ROI for an adipose characteristic. The adipose characteristic may include a measure of the tissue mechanical properties at one or more points, such as a tissue stiffness, a shear wave velocity between points, a longitudinal wave velocity between points, and a density of the tissue at one or more points. The adipose characteristic may also relate to thermal properties. Another adipose characteristic may be cavitation inducibility provided that non-adipose tissue would not be damaged under the conditions that produced cavitation in adipose tissue.

In addition to identifying the adipose tissue 104 and 106 by measuring for adipose characteristics, the diagnostic module 136 may identify other tissues by measuring for non-adipose characteristics. For example, the diagnostic module 136 may identify the connective tissue 108 and the muscle tissue 110 by measuring tissue mechanical properties at one or more points within the ROI, such as a tissue stiffness, a shear wave velocity between points, a longitudinal wave velocity between points, and a density of the tissue at one or more points. The non-adipose characteristic may also relate to thermal properties. Furthermore, techniques described above may be used to examine patterns in the image that are characteristic of different types of tissues (e.g., muscle tissue, connective tissue, bone tissue). For instance, a layered appearance characteristic of muscle tissue may be determined, or a cellular structure of adipose tissue caused by connective tissue boundaries could be determined.

As a more specific example, in some embodiments, the diagnostic module 136 may analyze images obtained through B-mode processing. A B-mode image is an image that may be gray-scale or colored showing a cross-section of the ROI along a scanning plane. The B-mode image relates to acoustic backscatter energy from different tissues within the ROI. FIG. 10 illustrates a B-mode gray-scale image of the ROI. As shown, different tissues may have different brightness levels. The diagnostic module 136 may measure a brightness level of a plurality of points within the image 102 to differentiate the adipose tissue 104 relative to other tissues. Furthermore, the diagnostic module 136 may use stored information, such as the expected anatomical structures for a particular ROI, in addition to the measured brightness levels to determine the adipose tissue 104. As one example, if the ROI is of the abdomen, the diagnostic module 136 may expect the image 102 to include the dermis layer 109 and the adipose tissues 104 and 106 separated by the connective tissue 108.

Also, the system 120 may use elastography processing methods alone or in addition to other processing methods to differentiate the adipose tissue 104 and 106. In one type of ultrasound elastography, a compression is applied to the ROI (e.g., along the skin surface) and strain images of the tissues in response to the compression may be obtained. For example, speckle tracking techniques may use data obtained before and after the applied compression to calculate the relative motion of different types of tissues due to their different mechanical properties (e.g., tissue strain or stiffness). As such, the images obtained through elastography, also called elastograms, relate to elastic properties of the tissues. Moreover, other techniques may be used to examine the mechanical properties of different tissues. Other elastography techniques, such as inducing and measuring the shear waves as described above or vibro-acoustography may be used to differentiate adipose tissue from other tissue types.

In some embodiments, after the initial scan to obtain diagnostic ultrasound signals has been performed and the adipose tissues 104 and 106 have been identified, the diagnostic module 136 may automatically set a therapy parameter before therapy is applied to the ROI. For example, the diagnostic module 136 may identify a thickness of a layer of the adipose tissue 104. Based on the thickness of the adipose tissue 104 and/or other factors, the diagnostic module 136 may automatically set a focal region depth, a focal region size, an ablation time for each point within the ROI that receives therapy, an energy level of the therapy signals, and a rate of focal region movement within the ROI during the therapy session. Other parameters may be automatically set, including a peak negative pressure, a pulse repetition rate, a duty cycle, a dwell time, and pulse sequences. Also, if the thickness, depth, or density of the layer of the adipose tissue 104 decreases or increases as the adipose tissue 104 extends laterally across the ROI (e.g., across the image 102), the therapy parameters may change in accordance with the thickness, density, or depth of the layer as the therapy is applied. For example, if the thickness is decreasing, then the focal region size may also decrease or the energy level of the therapy signals may decrease as the therapy moves along the adipose tissue 104.

After or while therapy is applied within the ROI, the diagnostic module 136 may analyze ultrasound signals from a second supplemental scan to confirm treatment of the ROI. For example, the diagnostic module 136 may obtain images from the second scan that include data processed through elastography methods, such as the shear-wave methods described above. After or while the adipose tissue 104 receives therapy, images obtained through elastography methods may indicate a change in tissue stiffness at a location that has received or is receiving therapy. More specifically, an initial image may have a color, brightness, or density that indicates a tissue stiffness of the adipose tissue 104 before treatment. In the second supplemental scan, the color, brightness, or density of the adipose tissue 104 may change where the adipose tissue 104 received therapy. The images from the initial and supplemental scans may be used by the system 120 to confirm that treatment was received within the adipose tissue 104. For example, the images from the initial and supplemental scans may be superimposed with each other or shown side-by-side on the display 138. The operator may confirm that treatment was received within the adipose tissue 104 by comparison of the images.

In addition or alternatively, the diagnostic module 136 may use other processing methods to facilitate determining whether a characteristic of the adipose tissue 104 or another tissue has changed due to therapy. Also, after evaluating the ROI in the second supplemental scan, the therapy parameters may be adjusted if therapy is to be applied again to the ROI or if the focal region moves to another location within the ROI.

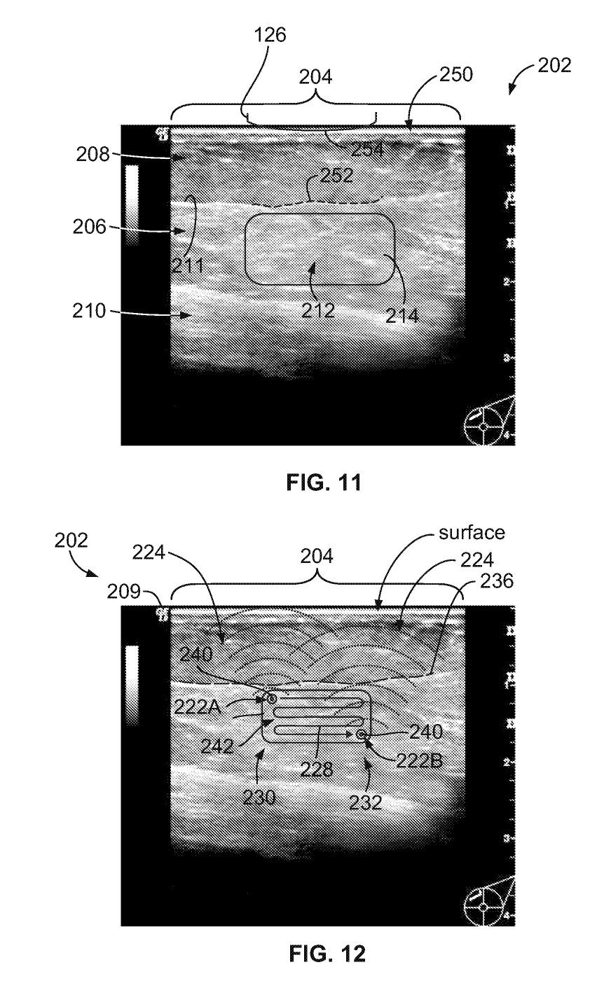

FIGS. 11 and 12 also illustrate features of the system 120 that may be used by an operator during a therapy session to facilitate delivering therapy to the ROI. FIG. 11 illustrates a window 202 that may be presented on the display 138 (FIG. 2). As shown in an image 204, the ROI includes adipose tissue 206 and 208 and non-adipose tissues, such as dermis layer 209, muscle tissue 210, and connective tissue 211. In some embodiments, the system 120 automatically designates a treatment space 212 or the user interface 142 (FIG. 2) accepts user inputs for designating the treatment space 212 within the ROI. The treatment space 212 represents a space that will be treated during a therapy session and is generally located within an adipose tissue, such as the adipose tissue 206. The designated treatment space 212 may correspond to a portion of the adipose tissue 206 within the image 204 or the treatment space 212 may correspond to all of the adipose tissue 206 within the ROI. By way of example, the treatment space 212 may be located and shaped so that the treatment space 212 is a distance away from the non-adipose tissues 211 and 210. As such, a probability of therapy being inadvertently applied to spaces outside of the treatment space 212, such as the non-adipose tissues 211 and 210, may be decreased.

The display 138 may indicate to the user or another viewer (e.g., the patient) the treatment space 212 designated by the user inputs. A graphical representation, such as an outline 214, may be overlaid upon the image 204. The outline 214 designates boundaries of the treatment space 212 to indicate to a viewer where the therapy will be applied. The outline 214 may be determined by parameters entered by the user. For example, the user may select pre-programmed outlines 214 or may enter coordinates or dimensions for the treatment space 212 to form the outline 214. The outline 214 may indicate an enclosed region within the treatment space 212. The outline 214 may have various shapes including a rounded rectangular shape (as shown), a parallelogram shape, another geometric shape, and the like, or a shape determined by the system 120.

The user may also enter a drawing notation to indicate where the outline 214 should be located. The drawing notation may be entered through a keyboard, a mouse, or another pointing device. As an example, the user may use a stylus pen and directly contact a touch-sensitive screen of the display 138 or a pad that is communicatively coupled to the user interface 142 to draw the drawing notation onto the image 204. As another example, the user interface 142 may recognize touches from a finger to the screen of the display 138. Furthermore, the user interface 142 may have a voice-activation module that receives voice commands from the user for entering user inputs including the drawing notation.

The outline 214 may be positioned with respect to reference points 250, 252, and 254. The reference module 195 (FIG. 3) may be configured to automatically identify the reference points 250, 252, or 254 on the patient or receive user inputs that identify the reference points. For instance, the reference point 250 may be a surface of the patient's skin or the dermis layer 209, the reference point 252 may be a particular point of or a portion of a boundary between the adipose tissues 206 and 208, and the reference point 254 may be a point along a surface of the probe 126. (For illustrative purposes, the reference point 254 and the probe 216 are shown in FIG. 11. However, the reference point 254 and the probe 216 may or may not be shown to the viewer in the image 204.) Reference points may also be other points within the ROI, such as bone, other artifacts, or a reference element such as a metallic sticker placed on a patient's skin.

After identifying a reference point, the reference module 195 may determine a relation of the treatment space 212 with respect to the identified reference point using ultrasound signal processing methods (e.g., speckle tracking). The reference module 195 may position the outline 214 of the treatment space 212 on the image 204 based on the relation of the treatment space 212 with respect to the identified reference point. As a more specific example, the reference module 195 may establish a positional relation between the adipose tissue 206 and the reference point 254 that represents a surface of the probe 126. Based on the positional relation, the reference module 195 may adjust a position of the treatment space 212 on the image 204. In other words, as the probe 126 moves along the surface of the skin or is pressed into the patient, the outline 214 on the image 204 may also move.

Furthermore, the reference module 195 may use data gathered or determined by the diagnostic module 136 (FIG. 2) to determine where to place the treatment space 212 within the ROI. For example, the reference module 195 may use information or data regarding the tissue characterization discussed above. More specifically, the reference module 195 may use the plurality of points within the ROI that were analyzed for adipose or non-adipose characteristics. Using this data, the reference module 195 may automatically locate or determine the position of the treatment space 212 within the ROI.

In some embodiments, the diagnostic module 136 may be configured to acquire the diagnostic ultrasound signals at different frame rates. A frame rate is the number of frames or images taken per second. More specifically, the diagnostic module 136 may be configured to acquire diagnostic ultrasound signals associated with different imaging spaces within the ROI at different frame rates. For example, signals from the treatment space 212 may be acquired at one frame rate while signals from other spaces outside of the treatment space 212 may be acquired at another frame rate. In one embodiment, the diagnostic module 136 is configured to acquire diagnostic ultrasound signals at a first rate in an imaging space that includes the treatment space 212 and at a slower second rate in an imaging space that excludes the treatment space 212. Alternatively, the first rate may be slower than the second rate.

FIG. 12 shows the window 202 as the system 120 (FIG. 2) delivers therapy to the treatment space 212. When therapy is applied, ultrasonic therapy signals (e.g., HIFU) from the probe 126 (FIG. 2) are directed toward a treatment location 222 (indicated as dots 222A and 222B in FIG. 12) within the treatment space 212. A treatment location 222 includes a region where a therapy beam 224 Formed by ultrasound signals from the transducer elements 124 is focused (e.g., a focal region of the transducer elements 124) within a body of a patient. The therapy beam 224 is shaped and directed by a selected configuration and operation of the transducer elements 124. As such, the focal region of the therapy beam 224 and, consequently, the treatment location 222 may vary in size and shape within a single therapy session. When the adipose tissue 206 is treated, the therapy beam 224 that is delivered to the treatment location 222 at least partially liquefies (e.g., through lysing, thermal damage, and/or cavitation) the adipose tissue 206 within the focal region.

The therapy module 125 (FIG. 2) is configured to move the treatment location 222 throughout the treatment space 212 between multiple points or treatment sites. As used herein, “moving the treatment location between multiple points” includes moving the treatment location 222 along a therapy path 228 between a first point and an end point and also includes moving the treatment location 222 to separate and distinct points within the treatment space 212 that may or may not be adjacent to one another along a path. The therapy path 228 may be formed by separate points where therapy is applied. For example, therapy may first be applied to a first point (indicated as the treatment location 222A). After therapy has been applied to the first point, the focal region may be readjusted onto a second point along the therapy path 228 that is separate and remotely spaced from the first point. Therapy may then be applied to the second point. The process may continue along the therapy path 228 until the therapy session is concluded at an end point (indicated as the treatment location 222B). In other embodiments, the therapy may be continuously applied as the focal region is moved along the therapy path 228 in a sweeping manner. For example, therapy may be continuously applied as the treatment location 222 is moved between the first point and the end point in FIG. 12.

Moreover, the therapy module 125 may operate in conjunction with the shear-wave-generating module 123 and the diagnostic module 136 to confirm that each treatment location 222 receives an effective amount of therapy. The system 120 may use the shear-wave methods described above. For example, after therapy has been applied to a first treatment location, the shear-wave-generating module 123 may automatically generate a shear wave at a generation site proximate to one side of the first treatment location. The diagnostic module may then automatically track a tracking site located on another side of the first treatment location to determine whether the treatment location received an effective amount of treatment. If the treatment location received an effective amount of treatment, the system 120 may then automatically move to a second treatment location.

The therapy path 228 may have various shapes and may be pre-programmed or, alternatively, drawn by the user. As shown in FIG. 12, the therapy module 125 may direct the treatment location 222 in a sweeping manner within the treatment space 212. More specifically, the treatment location 222 may move from a first lateral location 230 proximate one side of the image 204 or outline 214 to a second lateral location that 232 is proximate an opposing side of the image 204 or the outline 214. The treatment location 222 may maintain a predetermined depth within the adipose tissue 206 as the treatment location 222 moves between the first and second lateral locations 230 and 232. In some embodiments, after the treatment location 222 is moved from the first lateral location 230 to the second lateral location 232, the depth of the treatment location 222 may be increased or decreased. As shown in FIG. 12, the treatment location 222 moves back and forth between the first and second lateral locations 230 and 232 and increases a depth of the treatment location 222 after each crossing of the treatment space 212. As such, portions of the adipose tissue 206 may avoid sustaining multiple periods of therapy. Alternatively, the depth of the treatment location 222 may gradually change as the treatment location 222 is moved in a sweeping manner. As an example, the depth of the treatment location 222 within the adipose tissue 206 may move parallel to a boundary 236 (indicated as a dashed line) between the adipose tissues 206 and 208. The boundary 236 may or may not be shown to the viewer.

However, the therapy path 228 shown in FIG. 12 is just one example of applying therapy to multiple points within the treatment space 212. Many other therapy paths may be taken by the treatment location 222. For example, the therapy module 125 may direct the treatment location 222 in a sweeping manner between two vertical locations while changing a lateral position within the treatment space 212 after the vertical locations have been traversed. Furthermore, the treatment location 222 is not required to move between adjacent points along the therapy path 228, but may be moved to predetermined or random points within the treatment space 212 that are not adjacent to each other. For example, therapy may be applied to one corner of a treatment space 212. Subsequently, the focal region may then be readjusted to another corner and therapy may be applied.

As described above, the delivery of therapy may be based upon a therapy parameter. A therapy parameter includes any factor or value that may be determined by the system 120 or any input that may be entered by the user that affects the therapy applied to the ROI. For example, a therapy parameter may include a transducer parameter that relates to the configuration or operation of the transducer elements 124 or probe 126. Other examples of a transducer parameter include a focal region depth, a focal region size, an ablation time for each point within the ROI that receives therapy, an energy level of the therapy signals, and a rate of focal region movement within the ROI during the therapy session. Also, therapy parameters may include anatomical parameters, such as the location, shape, thickness, and orientation of the adipose tissue 206 and the non-adipose tissues. An anatomical parameter may also include a density of the adipose tissue 206 and the non-adipose tissues. Furthermore, therapy parameters include the type of probe 126 used during the therapy session. The age, gender, weight, ethnicity, genetics, or medical history of the patient may also be therapy parameters. After therapy has been applied to the treatment space 212, the system 120 or the operator may adjust the therapy parameters before applying therapy to the treatment space 212 again or another treatment space.

Returning to FIG. 12, in some embodiments, the display 138 may overlay another graphical representation, such as a marker 240, onto the image 204 that designates the treatment location or locations 222. The size and shape of the marker 240 may correspond to a size and shape of the focal region of the probe 126. As the therapy beam 224 moves the treatment location 222 within the treatment space 212, the display 138 may continuously update the marker 240 to cover new points within the treatment space 212 as the new points are receiving the therapy. In some embodiments, the marker 240 may only correspond to the point or points within the treatment space that are currently receiving treatment.

However, in other embodiments, the marker 240 or another graphical representation may also indicate a path within the treatment space 212 that has received therapy. For example, if the treatment location 222 is applied continuously and moved within the treatment space 212, the path may be indicated by a thick line (e.g., like a paint stroke) along the path. If the therapy is applied at separate and distinct points, a graphical representation, such as the marker 240, may be left on each point. As such, at an end of the therapy session, the image 204 may have multiple markers 240 overlaid upon the image 204 that indicate where therapy has been applied. In some embodiments, the graphical representations that indicate past therapy may remain on the image 204 indefinitely (i.e., until removed by the user or until the therapy session has concluded). In other embodiments, the graphical representations indicating past therapy may change as time progresses. Such graphical representations may indicate a time since therapy was applied, a fluidity of the tissue, a temperature, tissue stiffness, or some other characteristic of the tissue that may change with time. As an example, when therapy is first applied to a point, the graphical representation may be red to indicate that the point has recently received therapy. As time progresses, the graphical representation may fade or change into another color (e.g., blue) to indicate a predetermined amount of time has passed since therapy was applied to the point.

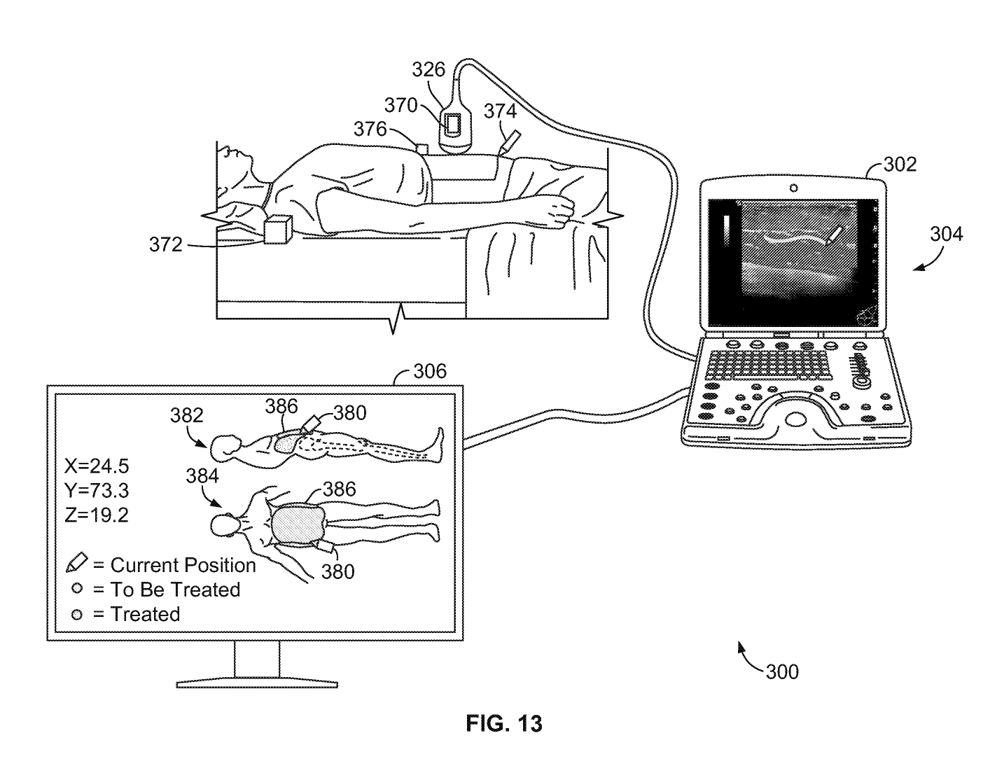

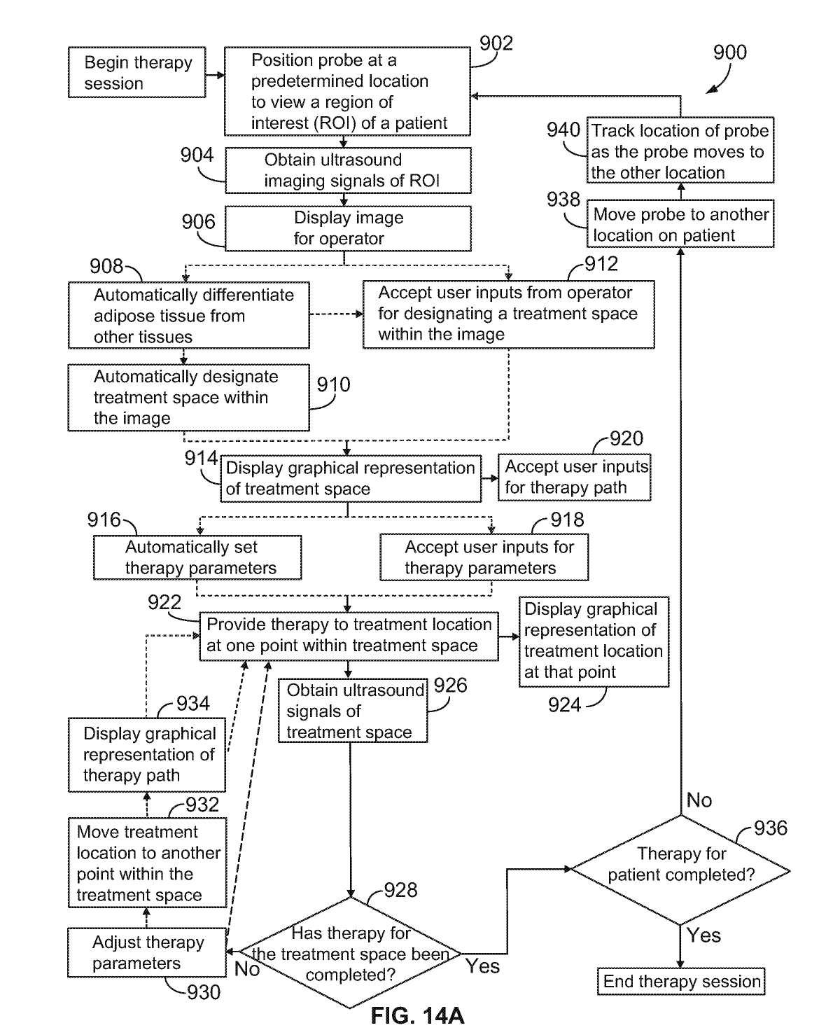

FIG. 13 illustrates an ultrasound system 300 formed in accordance with one embodiment. The system 300 may include similar features and components as described above with respect to FIGS. 1-11. More specifically, the system 300 includes a portable computer 302 that has a primary display 304 and that is communicatively coupled to a secondary display 306. The computer 302 may also include software and internal circuitry configured to perform as described above with respect to the system 120 (FIG. 2). The system 300 includes a probe 326 that is coupled to the computer 302 and has a probe position device 370. The system also includes a reference position device 372 that may be located near the patient or may be attached to the patient. The position devices 370 and 372 may have transmitters and/or receivers that communicate with each other and/or with the computer 302. For example, the position devices 370 and 372 may communicate with a position tracking module (not shown), such as the position tracking module 148 shown in FIG. 2. The position tracking module may receive signals from the position devices 370 and/or 372. In one particular embodiment, the position device 372 has a pair of coils that creates an electromagnetic field. The position tracking module receives data (e.g., positional information) from the position devices 370 and 372 regarding a location of the probe 326. As the probe 326 applies therapy to the patient and is moved along the patient, the display 304 and/or 306 may show the movement of the probe 326 with respect to the patient.

Also shown in FIG. 13, the system 300 may be configured to register where therapy will be applied during the therapy session. The system 300 may include an electronic pen 374 and fiducial element 376 attached to the body of the patient. The fiducial element 376 is attached near the sternum of the patient in FIG. 13, but may be attached to other spaces. A user desiring to outline or delineate where therapy will be applied may use the electronic pen 374 to draw on the body of the patient. First, the electronic pen 374 may register with the fiducial element 376 so that the location of the electronic pen 374 with respect to the body of the patient is known. After registering, the electronic pen 374 moves along the surface of the body and communicates with the computer 302 a current position of the electronic pen 374. Also, the electronic pen 374 may mark the patient's body (e.g., through ink, resin, or another substance) where therapy will be applied. The computer 302 uses the data received by the electronic pen 374 and the position device 372 to indicate on the display 306 where therapy is to be applied. As shown, the display 306 may show a graphical representation 382 of a side-view of the body and a graphical representation 384 of an anterior view of the body. The computer 302 uses the information from the electronic pen 374 to outline a region 386 of the body to be treated. The region 386 may be colored green prior to treatment. In an alternative embodiment, a single element or device may perform the functions of the fiducial element 376 and the reference position device 372.

As one example, the graphical representations 382 and 384 may be digital photographs of the patient's body. When therapy is applied to the body, the computer 302 tracks the position of the probe 326. As therapy is applied, the display 306 indicates an overall progress of the therapy session. For example, the display 306 may show the user the region of the body that is currently receiving therapy, the regions of the body that have already received therapy, and the regions of the body that have yet to receive therapy. For example, the regions that have received therapy may be colored red and the regions that have not received therapy may be colored green. Also, a graphical representation 380 of the probe 326 may be shown on the display 306 to indicate a current position of the probe 326 with respect to the body.

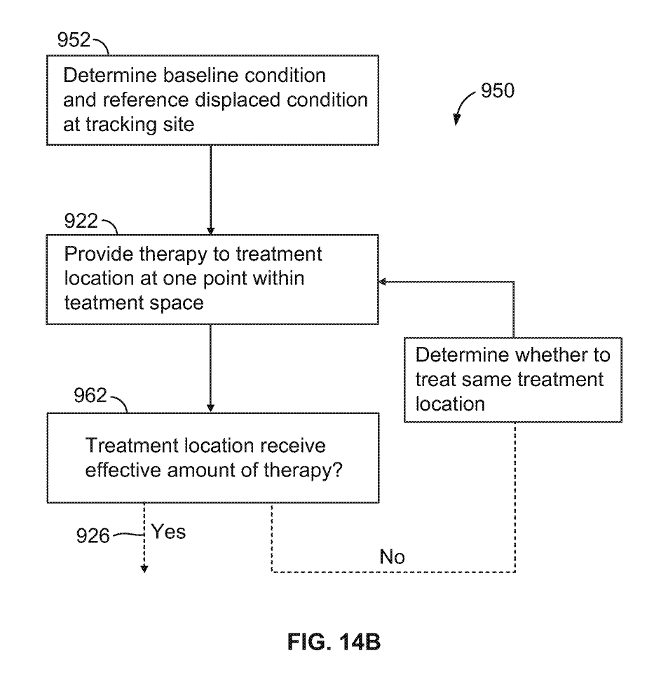

FIG. 14A is a flowchart illustrating a method 900 for delivering therapy to at least one ROI in a patient. The method 900 may be performed by a user or an operator of an imaging and therapy system. For example, the system used may be the systems 120, 300, or 450 (discussed above) or other systems described below. The therapy session may begin at 902 when the operator positions a probe at a predetermined location on the body of the patient to view an ROI. The ROI may be one of many that will be viewed during the therapy session. Ultrasound imaging signals of the ROI are obtained at 904. The signals may be processed into data via different ultrasound sub-modules, such as the modules 152-166 described above with reference to FIG. 2. In one embodiment, the signals are processed into data through at least one of B-mode and elastography processing.

An image of the ROI is generated and displayed to the operator and, optionally, patient at 906. The image may be, for example, a B-mode image in gray-scale and/or color. The image may also be a combination of images that are superimposed or arranged side-by-side with each other on a display. For example, the image may be formed from data obtained through B-mode processing and data obtained through elastography. Optionally, the system may automatically differentiate adipose tissue from other tissues at 908 and indicate to the operator the different layers of tissue within the image. The system may indicate the differentiation of tissues within the image to the user through lines, color, brightness, or other visual indications. Also, the system may automatically designate a treatment space at 910 by overlaying a graphical presentation (e.g., line) that indicates a boundary between the layers of tissue.

However, the system may also accept user inputs at 912 from the operator after a simple image (i.e., an image without graphical representations or other indications) of the ROI is displayed or after an image is displayed that automatically differentiates the tissues and indicates the different tissues to the operator. The user inputs may designate a treatment space. The system may display at 914 a graphical representation (e.g., an outline) of the designated treatment space. The system may then automatically set therapy parameters at 916 and/or the system may accept user inputs for therapy parameters at 918. For example, the system may determine mechanical properties of the treatment space by using the shear wave methods described above. Based upon the determined mechanical properties, the system may adjust the therapy parameters. Optionally, at 920, the operator may designate a therapy path within the treatment space. Therapy is then provided to a treatment location at 922 within the designated treatment space. The system may optionally, at 924, display a graphical representation (e.g., a marker) of the treatment location with the image.

After or while providing treatment to the treatment space, the system may obtain ultrasound signals of the treatment space at 926. At 928, the system determines whether treatment is complete. If treatment for the corresponding treatment space is not complete, the system may automatically adjust the therapy parameters at 930 and/or automatically move the treatment location at 932 to another point within the treatment space. The treatment location may move while providing treatment or after treatment has ended for a particular point. Furthermore, the therapy parameters may be adjusted while the treatment location is moving. Optionally, the system may display a graphical representation that indicates the path taken by the treatment location within the treatment space at 934. The system provides therapy to a new point and continues this process until the therapy for the corresponding treatment space is complete.

After therapy for the treatment space is completed, the system may determine (or ask the operator) at 936 whether therapy for the patient is complete. If therapy for the patient is complete, then the therapy session has ended. However, if the therapy session is not complete, then the system or the operator may move the probe at 938 to another location on the patient. In some embodiments, the system may also track at 940 a location of the probe as the probe moves to another location. The system may also display to the operator those regions that have already received treatment and those regions that have not received treatment.

Although the flowchart illustrates sequential steps in the method 900, embodiments herein include methods that perform fewer steps and also methods that perform the steps in different orders or may perform steps simultaneously. For example, the system may display an image of the ROI after the system has automatically differentiated the adipose tissue from other tissues. The system may also provide therapy to a treatment location within the ROI and simultaneously obtain imaging signals and display an image of the ROI during the therapy.

FIG. 14B is a flowchart illustrating a sub-method 950 for confirming an effective amount of therapy was delivered to a treatment location within an ROI of a patient. Before therapy is provided to a treatment location at 922, the system may automatically or may be commanded by the user to determine a baseline condition and a reference displaced condition at 952 of a tracking site located proximate to the treatment location. The baseline condition may be determined by tracking the tracking site when the adipose tissue is at rest. A shear wave may then be generated at a generation site that is proximate to the treatment location in which the treatment location is substantially between the generation and tracking sites. The reference displaced condition may be determined by tracking the tracking site when a shear wave propagates through the tracking site before the treatment location receives therapy. At 922, therapy is provided to the treatment location. The method 950 also includes determining at 962 whether the treatment location received an effective amount of therapy by propagating a shear wave from the generation site to the tracking site. The treatment location received an effective amount of therapy if the shear wave is not detected at the tracking site. The treatment location did not receive an effective amount of therapy if the shear wave is detected at the tracking site and the shear wave has not been substantially affected by propagating through the treatment location. If the treatment location did not receive an effective amount of therapy at the treatment location, then the system determines whether the same treatment location should be treated or whether a different treatment location located near the original treatment location should be treated. The system then provides therapy at 922 to the chosen treatment location. The therapy may be the same as before or include a different set of therapy parameters that may be more effective in treatment. If the original treatment location did receive an effective amount of therapy, the system moves on to step 926.

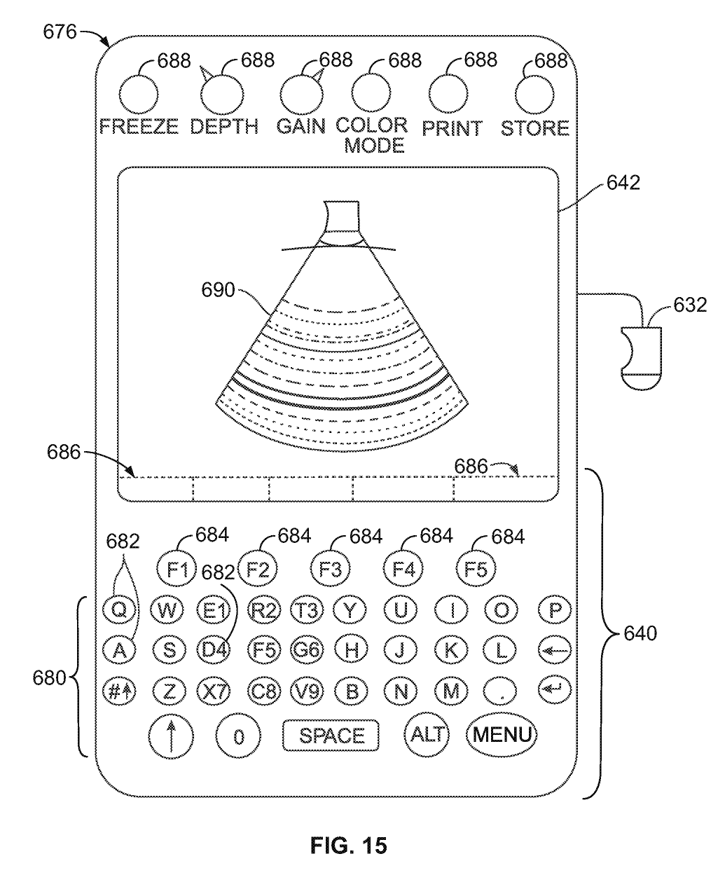

FIG. 15 shows another example of an ultrasound system and, in particular, a hand carried or pocket-sized ultrasound imaging system 676. In the system 676, a display 642 and a user interface 640 form a single unit. By way of example, the pocket-sized ultrasound imaging system 676 may be a pocket-sized or hand-sized ultrasound system approximately 2 inches wide, approximately 4 inches in length, and approximately 0.5 inches in depth and weighs less than 3 ounces. The display 642 may be, for example, a 320×320 pixel color LCD display (on which a medical image 690 may be displayed in combination with a graphical representation(s) as described above). A typewriter-like keyboard 680 of buttons 682 may optionally be included in the user interface 640. It should be noted that the various embodiments may be implemented in connection with a pocket-sized ultrasound system 676 having different dimensions, weights, and power consumption.

Multi-function controls 684 may each be assigned functions in accordance with the mode of system operation. Therefore, each of the multi-function controls 684 may be configured to provide a plurality of different actions. Label display spaces 686 associated with the multi-function controls 684 may be included as necessary on the display 642. The system 676 may also have additional keys and/or controls 688 for special purpose functions, which may include, but are not limited to “freeze,” “depth control,” “gain control,” “color-mode,” “print,” and “store.”

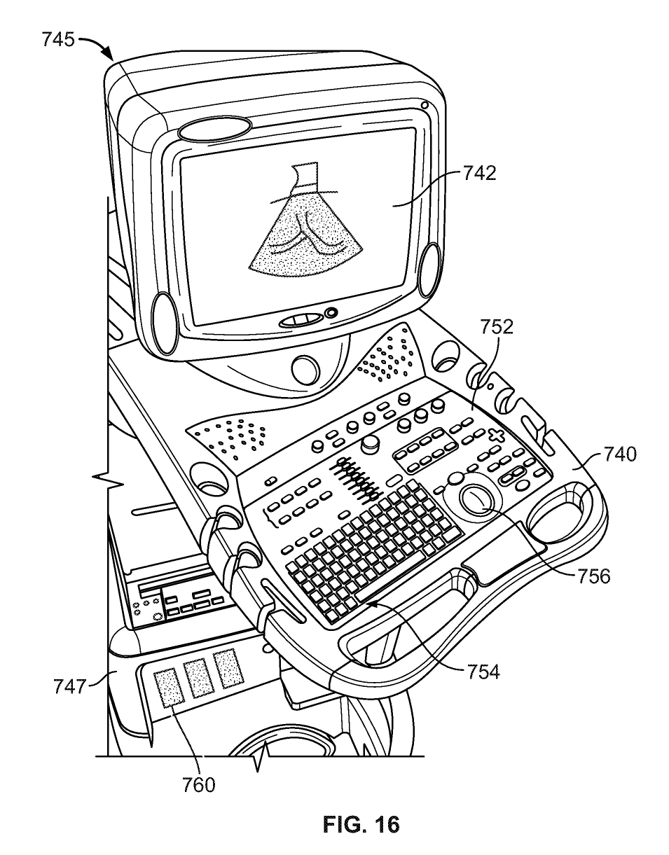

As another example shown in FIG. 16, a console-based ultrasound system 745 may be provided on a movable base 747 that may be configured to display the region of interest during a therapy session. The system 745 may also be referred to as a cart-based system. A display 742 and user interface 740 are provided and it should be understood that the display 742 may be separate or separable from the user interface 740. The user interface 740 may optionally be a touchscreen, allowing the operator to select options by touching displayed graphics, icons, and the like.

The user interface 740 also includes control buttons 752 that may be used to control the portable ultrasound imaging system 745 as desired or needed, and/or as typically provided. The user interface 740 provides multiple interface options that the user may physically manipulate to interact with ultrasound data and other data that may be displayed, as well as to enter user inputs and set and change imaging or therapy parameters. The interface options may be used for specific inputs, programmable inputs, contextual inputs, and the like. For example, a keyboard 754 and track ball 756 may be provided. The system 745 has at least one probe port 760 for accepting probes.

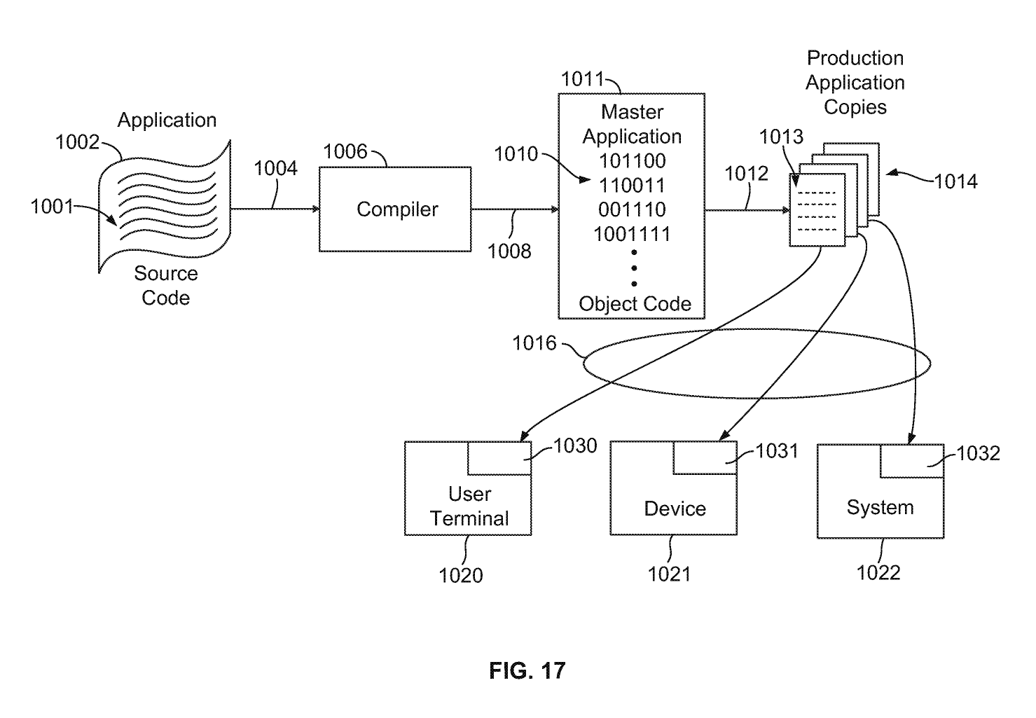

FIG. 17 is a block diagram of exemplary manners in which various embodiments described herein may be stored, distributed and installed on computer readable medium. In FIG. 17, the “application” represents one or more of the methods and process operations discussed above.

As shown in FIG. 17, the application is initially generated and stored as source code 1001 on a source computer readable medium 1002. The source code 1001 is then conveyed over path 1004 and processed by a compiler 1006 to produce object code 1010. The object code 1010 is conveyed over path 1008 and saved as one or more application masters on a master computer readable medium 1011. The object code 1010 is then copied numerous times, as denoted by path 1012, to produce production application copies 1013 that are saved on separate production computer readable medium 1014. The production computer readable medium 1014 is then conveyed, as denoted by path 1016, to various systems, devices, terminals and the like. In the example of FIG. 17, a user terminal 1020, a device 1021 and a system 1022 are shown as examples of hardware components, on which the production computer readable medium 1014 are installed as applications (as denoted by 1030-1032).

The source code may be written as scripts, or in any high-level or low-level language. Examples of the source, master, and production computer readable medium 1002, 1011 and 1014 include, but are not limited to, CDROM, RAM, ROM, Flash memory, RAID drives, memory on a computer system and the like. Examples of the paths 1004, 1008, 1012, and 1016 include, but are not limited to, network paths, the internet, Bluetooth, GSM, infrared wireless LANs, HIPERLAN, 3G, satellite, and the like. The paths 1004, 1008, 1012, and 1016 may also represent public or private carrier services that transport one or more physical copies of the source, master, or production computer readable medium 1002, 1011, or 1014 between two geographic locations. The paths 1004, 1008, 1012, and 1016 may represent threads carried out by one or more processors in parallel. For example, one computer may hold the source code 1001, compiler 1006 and object code 1010. Multiple computers may operate in parallel to produce the production application copies 1013. The paths 1004, 1008, 1012, and 1016 may be intra-state, inter-state, intra-country, inter-country, intra-continental, inter-continental and the like.

As used throughout the specification and claims, the phrases “computer readable medium” and “instructions configured to” shall refer to any one or all of i) the source computer readable medium 1002 and source code 1001, ii) the master computer readable medium and object code 1010, iii) the production computer readable medium 1014 and production application copies 1013 and/or iv) the applications 1030-1032 saved in memory in the terminal 1020, device 1021 and system 1022.

The various embodiments and/or components, for example, the monitor or display, or components and controllers therein, also may be implemented as part of one or more computers or processors. The computer or processor may include a computing device, an input device, a display unit, and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes described herein. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “Firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

Although the embodiments described above are illustrated as treating adipose tissue, alternative embodiments may be used to treat other tissues within the body. For example, the above described embodiments may be used to image and treat a tumor within a region of interest. As described above with respect to adipose tissue, embodiments may be used to automatically identify the tumor and/or to allow user inputs to identify treatment spaces within a region of interest and to set therapy parameters for the treatment. Furthermore, embodiments described herein may be used for palliative treatments for cancer, thermal treatment of muscles, or ultrasonically activating drugs, proteins, stem cells, vaccines, DNA, and gene delivery.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound system configured to determine whether adipose tissue of a patient received therapy at a treatment location, the system comprising:
an ultrasound probe;
a shear-wave-generating module to control the probe to provide a shear-wave beam that is configured to generate a shear wave at a first site within the patient the shear wave being configured to propagate through the treatment location toward a second site within the patient; and
a tracking module to control the probe to track the shear wave at the second site within the patient to determine if the treatment location received the therapy.

2. The system in accordance with claim 1 further comprising a therapy module to control the probe to deliver the therapy to the treatment location within the adipose tissue.

3. The system in accordance with claim 2 wherein the treatment location is a first treatment location located substantially between the first and second sites, the probe being controlled to deliver therapy to a second treatment location at the first site, the therapy at the second treatment location generating the shear wave that propagates toward the second site.

4. The system in accordance with claim 1 wherein the first site is located at the treatment location.

5. The system in accordance with claim 1 wherein the first site is located such that the treatment location is substantially between the first and second sites.

6. The system in accordance with claim 1 wherein the probe is configured to apply acoustic radiation force to the first site to generate the shear wave.

7. The system in accordance with claim 1 wherein the tracking module is configured to control the probe to deliver one or more tracking pulses to the second site.

8. The system in accordance with claim 1 further comprising a display to show a region-of-interest including the treatment location to an operator, the display displaying a graphical representation of the treatment location.

9. A method to determine whether adipose tissue of a patient received therapy at a treatment location, the method comprising:
providing a shear-wave beam to generate a shear wave at a first site within the patient, the shear wave being configured to propagate through the treatment location toward a second site within the patient; and
tracking the shear wave at the second site within the patient; and
determining whether the treatment location received the therapy.

10. The method in accordance with claim 9 wherein the determining includes determining that the treatment location had received an effective amount of the therapy if the shear wave has been substantially affected by propagating through the treatment location.

11. The method in accordance with claim 9 wherein the determining includes determining that the treatment location had not received an effective amount of the therapy if the shear wave is detected at the second site and has not been substantially affected by propagating through the treatment location.

12. The method in accordance with claim 9 wherein the generating the shear wave includes applying acoustic radiation force to the first site using focused ultrasound.

13. The method in accordance with claim 9 wherein the first site is at the treatment location.

14. The method in accordance with claim 9 wherein the first site is located such that the treatment location is substantially between the first and second sites.

15. The method in accordance with claim 9 wherein the tracking the shear wave includes using ultrasonic imaging techniques.

16. The method in accordance with claim 9 wherein the shear wave is a second shear wave, the method further comprising:
generating a first shear wave at the first site before delivering therapy to the treatment location; and
tracking the first shear wave at the second site, the first shear wave providing a reflection signal of the tracking pulse at the second site that is representative of the second site in a displaced condition.

17. The method in accordance with claim 16 further comprising determining mechanical properties of the adipose tissue between the first and second sites based upon the reflected signal of the second site in the displaced condition.

18. The method in accordance with claim 9 wherein the generating the shear wave at the first site and the tracking the shear wave at the second site are performed by a common ultrasound probe.

19. The method in accordance with claim 9 further comprising determining a baseline condition of the second site.

20. A system configured to determine whether adipose tissue of a patient received therapy at a treatment location, the system comprising:
a shear-wave-generator configured to generate a shear wave at a first site within the patient, the shear wave being configured to propagate through the treatment location toward a second site within the patient; and
a tracker configured to track the shear wave at the second site within the patient to determine if the treatment location received the therapy.

21. The system in accordance with claim 20 further comprising an ultrasound probe configured to deliver therapy to the treatment location within the adipose tissue.

22. The system in accordance with claim 20 wherein the tracker uses medical imaging techniques to track the shear wave at the second site.

23. The system in accordance with claim 21 wherein the probe comprises the shear-wave generator and the tracker.

24. The system in accordance with claim 22 wherein the treatment location is located at one of (a) the first site and (b) substantially between the first and second sites.
